(19) United States
(12) Patent Application Publication
(10) Pub. No.: US 2015/0119698 A1
(43) Pub. Date: Apr. 30, 2015

(54) Compositions Comprising Near-Infrared Fluorescent Particles And Uses Thereof For Imaging Activated Immune Cells In the CNS

(71) Applicants: Yissum Research Development Company of the Hebrew University of Jerusalem Ltd., Jerusalem (IL); Hadasit Medical Research Services and Development Ltd., Jerusalem (IL); Tel Hashomer Medical Research, Infrastructure And Service Ltd., Ramat Gan (IL); Drexel University, Philadelphia, PA (US)

(72) Inventors: Sara Eyal, Jerusalem (IL); Shlomo Magdassi, Jerusalem (IL); Emma Portnoy, Jerusalem (IL); Jacob Zauberman, Ramat Gan (IL); Boris Polyak, Philadelphia, PA (US); Jacob Golenser, Mevasseret Zion (IL); Yael Mardor, Netanya (IL); Dana Ekstein, Modi'in-Maccabim-Re'ut (IL)

(21) Appl. No.: 14/584,460

(22) Filed: Dec. 29, 2014

Related U.S. Application Data
(63) Continuation of application No. PCT/IL2013/050554, filed on Jun. 27, 2013.
(60) Provisional application No. 61/665,546, filed on Jun. 28, 2012.

(51) Int. Cl. A61K 49/00; A61B 5/00; A61B 5/055

(52) U.S. Cl. CPC A61K 49/0093; A61K 49/0017; A61K 49/0034; A61B 5/055; A61B 5/0071; A61B 5/4058
USPC 600/420; 600/431

(57) ABSTRACT

Pharmaceutical composition including nanoparticles configured for enhanced phagocytosis by phagocytic cells and labeled with a near-infrared (NIR) fluorescent probe bound to the outer surface thereof, and uses thereof in the detection of activated immune cells in the central nervous system (CNS) of a subject.

### FIELD OF THE INVENTION

The present invention relates to the use of nanoparticles labeled with a near-infrared (NIR) fluorescent probe for optical detection and imaging of activated immune cells in the central nervous system (CNS) of a subject.

### BACKGROUND OF THE INVENTION

A variety of disorders of the central nervous system (CNS), including for example multiple sclerosis, Alzheimer's disease, epilepsy, glioma, and cerebral malaria, are characterized by the presence of activated phagocytic cells within the CNS, either resident or blood-derived, invading, phagocytic cells (Prinz et al., 2011, Nature Neurosci, 14:1-9; Medana et al., 1997, Glia, 19:91-103; Sriram et al., 2011, J Neuroimmunol, 239:13-20; Zhai et al., 2011, Glia, 59:472-485; Malaguarnera et al., 2002, Lancet Infect Dis, 2:472-478; Vezzani el a., 2011, Nat Rev Neurol, 7:31-40; Zattoni et al., 2011, J Neurosci, 31:4037-4050; and Hanisch et al., 2007, Nat Neurosci, 10:1387-1394).

Tracking and imaging of sites of inflammation in a diseased CNS of a subject are desired. For example, such tracking and imaging may aid the diagnosis, as well as the evaluation of disease progression and effect of medical interventions, of various CNS disorders.

Known methods for brain imaging include, for example, magnetic resonance imaging (MRI) and positron emission tomography (PET). Stoll et al., 2010 Curr Opin Neurol, 23:282-286, review MRI-based techniques to visualize neuroinflammation in vivo, exemplified in multiple sclerosis and stroke. Assessment of brain inflammation after ischemic stroke using an ultra-small superparamagnetic particles of iron oxide (USPIO)-enhanced MRI has been described, for example in Nighoghossian et al., 2007, Stroke, 38(2):303-7; and Saleh et al., 2007, Stroke, 38:2733-2737. Vellinga et al., 2008, Brain, 131:800-807, describe the assessment of inflammation in multiple sclerosis by ultra-small iron oxide particle enhancement. However, MRI imaging methodology is considered expensive and interpretation of the hypointense signals requires highly experienced readers.

PET imaging with [¹¹C]-PK11195 and [¹¹C]-PBR28 has been applied to several CNS disorders, including Alzheimer's disease (Cagnin et al., 2001, Lancet, 358:461-467), Parkinson's disease (Gerhard et al., 2006, Neurobiol Dis., 21:404-412; and Ouchi et al., 2005, Ann Neurol, 57:168-175), and epilepsy (Butler et al, 2013, J Neuroimaging, 23(1):129-31, Epub 2011 Jan. 11; and Hirvonen et al., 2012, J Nucl Med, 53:234-240). However, PET is associated with health risks since it involves ionizing radiation, is technically demanding and is costly.

Indocyanine green (ICG) is a water-soluble cyanine fluorescent dye that absorbs and emits light in the near infrared (NIR) range. ICG is an FDA-approved molecule used for medical diagnostics, for example in determining cardiac output, hepatic function, liver blood flow, and ophthalmic angiography. The use of ICG as a contrast agent for imaging has been suggested for additional applications, reviewed, for example, in Marshall et al., 2010, Open Surg Oncol J., 2(2):12-25

In current clinical setups, ICG is used in aqueous solution as a free entity. Immobilization of ICG onto various surfaces has been described, for example, by embedding the ICG molecule within polymeric nanoparticles (Saxena et al., 2004, Int J Pharm, 278:293-301; Yaseen et al., 2009, Mol Pharm, 6:1321-1332; and Yu el al., 2010, J Am Chem Soc, 132:1929-1938), and by inclusion in liposomes and micelles (Devoiselle et al., 1997, Proc SPIE, 2980:530-537; Proulx et al., 2010, Cancer Res, 70:7053-7062; Sandanaraj et al., 2010, Bioconjug Chem, 21:93-101; and Kirchherr et al., 2009, Mol Pharm., 6(2):480-91).

The use of nanoparticles containing ICG probe and having a specific recognition to a targeted organ, system or tumor for in vitro and in vivo imaging has been described. Particular publications disclose, e.g., ICG injectable solution for checking the accuracy of cerebral blood flow measurements (Leung et al., 2007, Appl Opt., 46(10):1604-614), or for measuring blood flow in the retinal surface and sub retinal space of rabbit eyes (Maia et al., 2004, Retina., 24(1):69-79). Additional examples include cetuximab-labeled liposomes containing near-infrared probe for in vivo imaging (Portnoy et al., 2011, Nanomedicine (Lond), 7(4):480-8), and insoluble nanoparticles based on a cationic polymer, ICG and a targeting molecule or medical imaging (Larush et al., 2011, Nanomedicine (Lond), 6(2):233-40).

WO 2006/076636 discloses colloids containing polymer-modified core-shell particle carrier. More particularly, colloids containing core-shell nanoparticulate carrier particles are disclosed, wherein the shell contains a polymer having amine functionalities. The described carrier particles are stable under physiological conditions. The carriers can be bioconjugated with biological, pharmaceutical or diagnostic components.

WO 2007/025768 discloses, inter alia, nanoparticles having optically fluorescent activity. In more detail, a nanoparticle matrix is disclosed, comprising a co-aggregate of at least one charged polyelectrolyte and at least one oppositely charged active agent, wherein the active agent is a hydrophilic optically fluorescent agent. Further disclosed is a nanoparticle comprising said nanoparticle matrix.

US 2010/0183504 discloses a nanoparticle-based technology platform for multimodal in vivo imaging and therapy. In some embodiments, a probe comprising a nanoparticle coated with a hydrophilic coating attached to an imaging agent is provided. In some embodiments, the probe is used for the detection and/or treatment of a cancer.

US 2011/0280810 discloses a method of detecting a brain tumor which includes administering indocyanine green to a living body; exposing brain tissue in the living body; irradiating the exposed brain tissue with excitation light of indocyanine green; obtaining an image based on fluorescence of the excited indocyanine green in the brain tissue, wherein the image is obtained using an endomicroscope; and identifying portions of the brain tissue corresponding to the brain tumor based on the image.

WO 2012/032524, to some of the inventors of the present invention, discloses particles comprising either a water-insoluble polymer or a phospholipid, wherein at least one near infrared (NIR) fluorescent probe and optionally at least one active agent such as a targeting moiety, capable of selectively recognizing a particular cellular marker, are non-covalently bound to the outer surface of the particles. It is disclosed that pharmaceutical compositions comprising these particles may be used, inter alia, for detection and treatment of tumors in the gastrointestinal tract.

None of the art discloses or suggests imaging of activated immune cells in the CNS of a subject using particles labeled with a NIR fluorescent probe, such as ICG. In particular, nowhere is it disclosed or suggested that such labeled particles can be designed for efficient uptake by activated phagocytic cells in inflamed areas of the CNS during neuroinflammation, resulting in a clear and specific fluorescent signal that can be used for tracking the areas of inflammation. There is a medical need for compositions and methods for simple and accurate imaging of areas of inflammation in the CNS, which can be useful, for example, for the diagnosis, evaluation of disease state and monitoring response to treatment of various CNS disorders.

### SUMMARY OF THE INVENTION

The present invention provides pharmaceutical compositions comprising nanoparticles labeled with a near-infrared (NIR) fluorescent probe, and methods for detecting activated immune cells in the central nervous system (CNS) of a subject using the same.

The present invention discloses for the first time that activated immune cells, particularly myeloid cells such as phagocytic cells, in the CNS of a subject having a disease where CNS inflammation is involved can be detected and visualized in vivo by optical means. The detection can be performed, according to some embodiments, by systemically administering to the subject nanoparticles comprising a NIR fluorescent probe, irradiating at least a portion of the CNS with excitation radiation of the probe, and collecting NIR signals emitted from the probe. According to some embodiments, the nanoparticles are uptaken by phagocytic cells at the areas of inflammation in the CNS, either resident or blood-derived, invading phagocytic cells, and accumulate in these areas. Upon excitation of the probe with NIR radiation, the locality of activated phagocytic cells, and therefore of areas of inflammation, can be identified and imaged by detecting NIR fluorescence emission from the probe.

As exemplified hereinbelow in a mouse model of cerebral malaria, administration of fluorescently labeled nanoparticles to infected mice resulted in a clear fluorescent signal from the brain of the mice compared to naïve mice that were similarly administered with labeled nanoparticles, indicating preferred uptake of labeled nanoparticles into the brain of infected compared to naïve mice. Administration of the probe in a free form to infected mice did not result in significant fluorescence from the mice brain. As further exemplified hereinbelow, confocal microscopy experiments performed in a mouse model of epilepsy showed co-localization of labeled nanoparticles mainly with microglia/macrophages in areas of brain inflammation.

Advantageously, the nanoparticles according to embodiments of the present invention are characterized by one or more structural and physicochemical features that increase their uptake by phagocytic cells, thereby enhancing the fluorescent signal from the inflammation regions to facilitate better detection. For example, the nanoparticles may be sized such that their uptake is increased. Alternatively or additionally, the nanoparticles may be charged, either negatively or positively, and/or comprise surface ligands that target the nanoparticles to phagocytic cells.

The compositions and methods of the present invention are particularly beneficial as they allow simple detection of areas of inflammation in the CNS using optical means, with possible real-time imaging.

According to one aspect, the present invention provides a pharmaceutical composition comprising nanoparticles comprising a near-infrared (NIR) fluorescent probe and a pharmaceutically acceptable carrier, for use in the detection of activated immune cells in the central nervous system (CNS) of a subject.

In some embodiments, a pharmaceutical composition is provided, comprising: (i) nanoparticles configured for enhanced phagocytosis by phagocytic cells, characterized by at least one structural or physicochemical feature that enhances their uptake by phagocytic cells compared to equivalent nanoparticles without the at least one feature, the nanoparticles further comprise a near-infrared (NIR) fluorescent probe; and (ii) a pharmaceutically acceptable carrier; for use in the detection of activated phagocytic cells in the central nervous system (CNS) of a subject.

In some embodiments, a pharmaceutical composition is provided, comprising: (i) nanoparticles comprising at least one targeting moiety that targets the nanoparticles to the outer surface of phagocytic cells, the nanoparticles further comprise a near-infrared (NIR) fluorescent probe; and (ii) a pharmaceutically acceptable carrier; for use in the detection of activated phagocytic cells in the central nervous system (CNS) of a subject.

In some embodiments, the size of the nanoparticles is in the range of about 80 nm-20 microns. In some embodiments, the size of the nanoparticles is in the range of about 80 nm-1000 nm.

In some embodiments, the nanoparticles are charged. In some embodiments, the nanoparticles are negatively charged. In other embodiments, the nanoparticles are positively charged.

In some embodiments, the nanoparticles comprise at least one targeting moiety bound to the outer surface thereof that targets the nanoparticles to phagocytic cells.

In some embodiments, the targeting moiety that targets the nanoparticles to phagocytic cells is selected from the group consisting of a peptide, protein, antibody, lectin, polysaccharide, glycolipid, and glycoprotein.

In some embodiments, the targeting moiety is non-covalently bound to the outer surface of the nanoparticles. In other embodiments, the targeting moiety is covalently bound to the outer surface of the nanoparticles.

In some embodiments, the nanoparticles are characterized by at least one of: size in the range of about 80 nm-20 microns (or in the range of about 80 nm-1000 nm), charge (either negative or positive) and a surface-bound targeting moiety that targets the nanoparticles to phagocytic cells.

In some embodiments, the NIR fluorescent probe is selected from the group consisting of a fluorescent dye and NIR quantum dots.

In Particular Embodiments, the NIR Fluorescent Probe is Indocyanine Green (ICG).

In some embodiments, the NIR fluorescent probe is bound to the outer surface of the nanoparticles. In some embodiments, the NIR fluorescent probe is non-covalently bound to the outer surface of the nanoparticles. In other embodiments, the NIR fluorescent probe is covalently bound to the outer surface of the nanoparticles.

In some embodiments, the NIR fluorescent probe is embedded within the nanoparticles.

In some embodiments, the nanoparticles comprise up to about 10% (w/w) of the NIR fluorescent probe.

In some embodiments, the nanoparticles further comprise at least one magnetic probe detectable by magnetic resonance imaging (MRI).

In some embodiments, the magnetic probe is non-covalently bound to the outer surface of the nanoparticles. In other embodiments, the magnetic probe is covalently bound to the outer surface of the nanoparticles.

In some embodiments, the nanoparticles are capable of penetrating the blood-brain-barrier (BBB).

In some embodiments, the nanoparticles are liposome nanoparticles.

In additional embodiments, the nanoparticles are polymeric nanoparticles, wherein one or more polymers form the core, or matrix, of the nanoparticles.

In yet additional embodiments, the nanoparticles are solid lipid nanoparticles.

In some embodiments, the pharmaceutical composition is formulated for systemic parenteral administration.

In some embodiments, the pharmaceutical composition is formulated for a route of administration selected from the group consisting of intravenous, intraarterial, trans-nasal, intrathecal, and intra-orbital.

In some embodiments, the concentration of the nanoparticles in the composition is in the range of about 0.01-10% (w/w).

According to another aspect, the present invention provides a method for detecting activated immune cells, particularly myeloid cells, such as macrophages, in the CNS of a subject.

In some embodiments, the method comprises the steps of: (i) parenterally administering to a subject a pharmaceutical composition of the present invention; (ii)

irradiating at least a portion of the CNS of the subject with NIR radiation having a wavelength that is absorbed by the NIR fluorescent probe; and (iii) detecting NIR fluorescence emission from the probe, wherein a locality of said fluorescence emission from the probe is indicative of a locality of activated phagocytic cells, thereby detecting activated phagocytic cells in the CNS of the subject.

In some embodiments, the method comprises the steps of: (i) irradiating at least a portion of the CNS of a subject pre-administered with a pharmaceutical composition of the present invention with NIR radiation having a wavelength that is absorbed by the NIR fluorescent probe; and (ii) detecting NIR fluorescence emission from the probe, wherein a locality of said fluorescence emission from the probe is indicative of a locality of activated phagocytic cells, thereby detecting activated phagocytic cells in the CNS of the subject.

In some embodiments, the method comprises the step of: detecting NIR fluorescence emission of a NIR fluorescent probe from a portion of the CNS of a subject following parenteral administration of a pharmaceutical composition of the present invention and irradiation of said portion of the CNS of a subject with NIR radiation that is absorbed by the probe, wherein a locality of said fluorescence emission from the probe is indicative of a locality of activated phagocytic cells, thereby detecting activated phagocytic cells in the CNS of the subject.

In some embodiments, detecting comprises obtaining one or more images of the portion of the CNS irradiated by NIR where areas of NIR fluorescent emission are indicated.

In some embodiments, detecting comprises detecting using a microscope with appropriate filters.

In some embodiments, the NIR fluorescent probe is selected from the group consisting of a fluorescent dye and NIR quantum dots.

In some embodiments, the NIR fluorescent probe is ICG.

In some embodiments, the NIR radiation has a wavelength in the range of about 700-850 nm.

In some embodiments, the subject is having, or suspected of having, a disease associated with CNS inflammation.

In some embodiments, the disease associated with CNS inflammation is selected from the group consisting of epilepsy, cerebral malaria, cysticercosis, lupus, multiple sclerosis, autoimmune encephalomyelitis, stroke, glioma, Alzheimer's disease, Parkinson's disease, traumatic brain injury, autism, and schizophrenia.

In some embodiments, the method is used for the detection of an area of inflammation in the brain of the subject.

In some embodiments, the pharmaceutical composition is administered via a route of administration selected from the group consisting of intravenous, intraarterial, trans-nasal, intrathecal, and intra-orbital.

In some embodiments, the detection is performed several minutes up to several hours following administration of the pharmaceutical composition.

These and further aspects and features of the present invention will become apparent from the detailed description, examples and claims which follow.

### BRIEF DESCRIPTION OF THE FIGURES

FIG. 1. Uptake of non-PEGylated NP (left) and PEGylated NP (right) liposome nanoparticles labeled with ICG by RAW 264.7 macrophages.

FIGS. 2A-2C. Distribution of ICG (free or bound to liposome nanoparticles) to the CNS in a murine model of cerebral malaria and in naïve controls. (A) In vivo, infected mice versus control, free or liposome-bound ICG; (B) In vivo, free versus liposome-bound ICG in infected mice; (C) In vitro, free versus liposome-bound ICG in infected mice brain tissue.

FIGS. 3A-3B. Intensity of ICG emission from brain compared to foot following administration of liposome nanoparticles labeled with ICG in mice infected with Plasmodium berghei ANKA (A) versus naïve controls (B).

FIG. 4. Brain scans following ICG administration (free or nanoparticle-bound) of mice infected with P. berghei ANKA and naïve controls.

FIGS. 5A-5B. Characterization of magneto-NP by high resolution scanning electron microscopy (HR-SEM) (A), and transmission electron microscopy (TEM) (B).

FIGS. 6A-6C. Confocal microscope images of an exemplary epileptic rat brain slice focused on epileptogenic brain region—hippocampus. (A) Stained brain slice, “+” sign indicates the brain region illustrated in B-C; (B) Merged image, microglia/macrophages (dashed circles), astrocytes (solid-line circles), DAPI and nanoparticles (dashed arrows); (C) nanoparticles only.

FIGS. 7A-7C. Confocal microscope images of an exemplary epileptic rat brain slice focused on epileptogenic brain region—hippocampus. (A) Stained brain slice, “+” sign indicates the brain region illustrated in B-C; (B) Merged image, microglia/macrophages (solid-line circle), endothelial cells (dashed squares), DAPI and nanoparticles (dashed circles); (C) nanoparticles only.

FIGS. 8A-8D. Confocal microscope images of an exemplary epileptic rat brain slice focused on the thalamus. (A) Stained brain slice, “+” sign indicates the brain region illustrated in B-D; (B) Merged image, microglia/macrophages, endothelial cells (circles), DAPI and nanoparticles (dashed arrows); (C) Nanoparticles only; (D) Merged image, nanoparticles (dashed arrows) and microglia/macrophages.

FIGS. 9A-9C. Confocal microscope images of exemplary brain slices of epileptic rats sacrificed 4 h post injection of nanoparticles and brain slices of naïve rats sacrificed 4 h post injection of nanoparticles. (A) Stained brain slice, “+” sign indicates the brain region illustrated in B-C; (B) Merged image, microglia/macrophages (circles), nanoparticles (dashed arrows) and DAPI in a naïve rat; (C) Merged image, microglia/macrophages (circles), nanoparticles (dashed arrows) and DAPI in an epileptic rat.

FIG. 10. Uptake of neutral versus negatively charged PLA-based particles by murine macrophages (RAW 264.7).

### DETAILED DESCRIPTION OF THE INVENTION

The present invention is directed to optical imaging of areas of inflammation in the CNS of a subject using nanoparticles comprising a NIR-fluorescent probe. The nanoparticles are configured such that their uptake by phagocytic cells is enhanced. In some embodiments, the nanoparticles are configured such that they are targeted to the outer surface of phagocytic cells.

Pharmaceutical Compositions

The pharmaceutical compositions of the present invention comprise biocompatible, nanoparticles that arc fluorescent in the near infrared (NIR) range and configured for enhanced phagocytosis by phagocytic cells, such as peripheral, circulating, phagocytic cells including monocytes and macrophages, and/or CNS resident phagocytic cells including microglia. The term “biocompatible” as used herein indicates that the particles are made of compounds suitable for administration, including intravenous administration, to mammals, including humans.

The nanoparticles are characterized by at least one structural or physicochemical feature that enhances their uptake by phagocytic cells compared to equivalent nanoparticles without the at least one feature. In some embodiments, the feature is size. In additional embodiments, the feature is charge. In yet additional embodiments, the feature is the presence of a surface-bound ligand that targets the nanoparticles to phagocytic cells of the immune system. In yet additional embodiments, the feature is the absence of surface modifications that prolong the lifetime of particles in the circulation, such as PEGylation.

The size of the nanoparticles of the present invention may range between about 80 nm-20 microns, for example from about 80 nm-5 microns, about 80 nm-2 microns, about 80 nm-1 micron. Thus, although referred to herein as “nanoparticles”, micron scale particles are also encompassed. For intravenous or intra-arterial injection administration, the size of the particles is preferably in the range of about 80 nm-1000 nm. For other routes of administration, for example trans-nasal, larger particles may be used. In some embodiments, particles with a size in the range of about 20-300 nm, for example about 20-100 nm, 20-50 nm are used. Each possibility represents a separate embodiment of the invention.

As used herein, the term “about”, when referring to a measurable value such as an amount or size, is meant to encompass variations of +/−10%, more preferably +/−5%, even more preferably +/−1%, and still more preferably +/−0.1% from the specified value, as such variations are appropriate to achieve the intended purpose.

As used herein, the “size” of the nanoparticles indicates that the longest dimension of the nanoparticles (width, length or diameter) is in the specified range. Typically, the average particle size in a preparation comprising the nanoparticles is in the specified range. The nanoparticles may be of a uniform shape, e.g., spherical or elongated, or may have a variety of shapes.

The nanoparticles of the present invention may be negatively or positively charged. As used herein, the “charge” of the nanoparticles refers to their surface charge, known as zeta potential. For intravenous or intraarterial administration, negatively charged particles are currently preferred. The range of surface charge (zeta potential) for negatively charged particles may range from about −20 to −55 mV.

Particle size and zeta-potential measurements can be performed by methods known in the art, for example, by dynamic light scattering (DLS) using commercially available instruments, e.g. a Zetasizer NanoZS (Malvern, UK).

In some embodiments, the nanoparticles of the present invention comprise at least one targeting moiety bound to the outer surface thereof that targets the nanoparticles to phagocytic cells, thereby enhancing phagocytosis of the particles. In some embodiments, the nanoparticles comprise at least one targeting moiety bound to the outer surface thereof that targets the nanoparticles to the outer surface of phagocytic cells, and mediates their binding to phagocytic cells. In some embodiments, the targeting moiety can be selected such that additional types of myeloid cells (which are not phagocytic) are targeted. According to these embodiments, myeloid cells, including phagocytic and non-phagocytic, in the areas of inflammation in the CNS of a subject can be detected.

The targeting moiety may be non-covalently or covalently bound to the outer surface of the nanoparticles. Each possibility represents a separate embodiment of the invention.

The targeting moiety that targets the nanoparticles to phagocytic cells may include a peptide, a protein, an antibody, a lectin, a polysaccharide, a glycolipid, and a glycoprotein. Each possibility represents a separate embodiment of the present invention.

Examples of suitable targeting moieties are described, for example, in Kelly et al., 2011, J Drug Deliv., 2011:727241, and include, but are not limited to, muramyl tripeptide (MTP), Arg-Gly-Asp (RGD), Anti-VCAM-1, Anti-CC52, Anti-CC531, Anti-CD11c/DEC-205, Mann-C4-Chol, Man2DOG, Aminophenyl-α-D-mannopyranoside, Man3-DPPE, maleylated bovine serum albumin (MBSA), O-steroly amylopectin (O-SAP), fibronectin and galactosyl. Each possibility represents a separate embodiment of the present invention.

In some embodiments, the targeting moiety is an immunoglobulin G (IgG). In some embodiments, the nanoparticles are surface-functionalized with IgG without chemical modifications (preparation is based on physicochemical interactions without covalent bonds). Functionalization with IgG is aimed at facilitating phagocytosis of the nanoparticles due to interaction with Fc receptors known to be highly expressed on the surface of myeloid cells (Kettenmann et al., 2011, Physiol Rev., 91:461-553; Moghimi et al., 2001, Grit Rev Ther Drug Carrier Syst., 18:527-550; Moghimi et al., 2003, Prog Lipid Res,42:463-478).

In some embodiments, the nanoparticles of the present invention are capable of penetrating the blood-brain-barrier.

The nanoparticles of the present invention may include liposome nanoparticles, polymer nanoparticles, or solid lipid nanoparticles.

In some embodiments, the nanoparticles of the present invention are liposomes.

Liposomes for use in this invention may be prepared to include liposome-forming lipids and phospholipids, and membrane active sterols (e.g. cholesterol). Liposomes may include other lipids and phopsholipids which are not liposome forming lipids.

Phospholipids may be selected, for example, from a lecithin (such as egg or soybean lecithin); a phosphatidylcholine (such as egg phosphatidylcholin); a hydrogenated phosphotidylcholine; a lysophosphatidyl choline; dipalmitoylphosphatidylcholine; distearoylphosphatidylcholine; dimyristoylphosphatidylcholine; dilauroylphosphatidylcholine; a glycerophospholipid (such as phosphatidylglycerol, phosphatidylserine, phosphatidylethanolamine, lysophosphatidylethanolamine, phosphatidylinositol, phosphatidylinositol phosphate, phosphatidylinositol bisphosphate and phosphatidylinositol triphosphate); sphingomyelin; cardiolipin; a phosphatidic acid; a plasmalogen; or a mixture thereof. Each possibility represents a separate embodiment of the invention.

Examples of other lipids that can be used include a glycolipid (such as a glyceroglycolipid, e.g. a galactolipid and a sulfolipid, a glycosphingolipid, e.g., a cerebroside, a glucocerebroside and a galactocerebroside, and a glycosylphosphatidylinositol); a phosphosphingolipid (such as a ceramide phosphorylcholine, a ceramide phosphorylethanolamine and a ceramide phosphorylglycerol); or a mixture thereof. Each possibility represents a separate embodiment of the invention.

Negatively or positively charged liposome nanoparticles can be obtained, for example, by using anionic or cationic phospholipids or lipids. Such anionic/cationic phospholipids or lipids typically have a lipophilic moiety, such as a sterol, an acyl or diacyl chain, and where the lipid has an overall net negative/positive charge.

The above described lipids and phospholipids can be obtained commercially or prepared according to published methods in the art.

Liposomes can be prepared by methods known in the art, reviewed, for example, in Scholar et al., 2012, International Journal of Pharmaceutical Studies and Research, 3(2): 14-20; Akbarzadeh et al., 2013, Nanoscale Research Letters, 8:102. Exemplary procedures are described hereinbelow. Extrusion of liposomes through a small-pore membrane, e.g. polycarbonate membrane, is an effective method for reducing liposome size down to a relatively well-defined size distribution. Typically, the suspension is cycled through the membrane several times (using membranes of decreasing pore sizes) until the desired liposome size distribution is achieved. The liposomes extrusion through successively smaller-pore membranes, enables a gradual reduction in liposome size down to the desired size. The down-sized processed liposome suspension may be readily sterilized by passage through a sterilizing membrane having a particle discrimination size of, e.g, about 0.2 microns, such as a conventional 0.22 micron depth membrane filter. If desired, the liposome suspension can be lyophilized in the presence of a suitable cryoprotectant for storage and reconstituted by hydration shortly before use.

In some embodiments, the nanoparticles of the present invention are polymer nanoparticles.

The polymer-based nanoparticles may include a concentrated core containing a probe (such as a magnetic probe) surrounded by a polymeric shell. Alternatively, one or more polymers may form a matrix in which a probe is embedded. Yet another alternative is where one or more polymers form the core of the nanoparticles, while fluorescent and/or magnetic probes are attached to the outer surface of the nanoparticles.

The polymers for use in the present invention may include synthetic or natural water-insoluble polymers. Examples of natural polymers include proteins, polysaccharides and lipids, as described, e.g., in Quintanar-Guerrero et al., 1998, Drug Dev Ind Pharm, 24:1113-28; and Kumar et al., 2000, J Pharm Pharmaceut Sci, 3:234-58).

Examples of synthetic polymers include poly(ester)s, poly(urethane)s, poly(alkylcyanocrylate)s, poly(anhydride)s, poly(ethylenevinyl acetate), poly(lactone)s, poly(styrene)s, poly(amide)s, poly(acrylonitrile)s poly(acrylate)s, poly(methacrylate)s, poly(orthoester)s, poly(ether-ester)s, poly(tetrafluoroethylen)s, mixtures of thereof and copolymers of corresponding monomers.

In certain embodiments, the poly(ester) is a member selected from the group consisting of poly(lactic acid), poly(glycolic acid), poly(lactic-co-glycolic acid), poly(c-caprolactone), poly(dioxanone), poly(hydroxybutyrate), and poly(ethylene terephthalate).

The polymers suitable for use according to embodiments of the present invention are biocompatible, and are not immunogenic, mutagenic, thrombogenic (i.e. cause blood coagulation or clotting) or toxic (including the polymer degradation products).

Polymeric nanoparticles can be prepared by methods known in the art, for example, as described in Saxena et al., 2004 noted above; Yaseen et al., 2009 noted above; Yu el al., 2010 noted above; Vauthier et al., 2009, Pharmaceutical Research, 26(5): 1025-1058; and in the “Nanoparticle Technology Handbook”, 2012, edited by Kiyoshi Nogi, Masuo Hosokawa, Makio Naito, Toyokazu Yokoyama, Elsevier. Exemplary procedures are described hereinbelow.

The size and charge of polymeric particles can be controlled by methods known in the art. For example, for poly(lactic acid)-based particles, the size of the particles can be controlled by adjusting the ratio of the organic solvents used in the emulsification step. The inclusion of a water-miscible solvent (e.g. tetrahydrofuran, THF) in the organic phase results in a decrease in particle size as its gradient-driven distribution into the aqueous medium provides additional energy resulting in formation of smaller sized nanospheres. The surface charge of polymeric particles can be controlled by the stabilizing polymer used in the emulsification step (Chorny et al., 2007, FASEB J, 21:2510-9).

In some embodiments, the nanoparticles of the present invention are formed from non-polymeric substances that form the particle matrix, such as solids. Thus, in some embodiments, the nanoparticles of the present invention are solid lipid nanoparticles. Examples of suitable solid lipids for preparing such nanoparticles include glycerides and fatty acids. The solid lipid particles could be prepared, for example, by solvent emulsification-diffusion method (Trotta et al., 2003, Int J Pharm, 257:153-60) nanoemulsion method (Mao et al., 2003, Yao xue xue bao=Acta pharmaceutica Sinica, 38:624-6), high pressure homogenization, ultrasonication, solvent emulsification/evaporation, microemulsion, spray drying and double emulsion method (Mulla et al., 2011, Indian Journal of Novel Drug delivery, 3(3): 170-175; Li et al., May 10, 2013 [Epub ahead of print], Drug Dev Ind Pharm; Morsi et al., 2013, Pharmaceutical development and technology., 18:736-44; Parhi et al., 2012, Current drug discovery technologies, 9:2-16; Noriega-Pelaez et al., 2011, Drug Dev Ind Pharm., 37:160-6; Gallarate et al., 2009, J Microencapsul., 26:394-402; Li et al., 2006, Zhongguo yi xue ke xue yuan xue bao=Acta Academiae Medicinae Sinicae, 28:686-9; Hu et al., 2004, Int J Pharm, 273:29-35; and Zhang et al., 2003, Yao xue xue bao=Acta pharmaceutica Sinica, 38:302-6).

The nanoparticles of the present invention comprise at least one near-infrared (NIR) fluorescent probe bound to their outer surface. In some embodiments, the binding is non-covalent. In other embodiments, the binding is covalent.

As used herein, a “near-infrared (NIR) fluorescent probe” is a molecule or entity suitable for imaging applications, capable of absorbing and emitting light in the NIR spectral range. In particular, it is a fluorescent entity having an excitation light and emission light in the NIR spectral range, preferably in the range of about 700 to 900 nm. NIR radiation is typically defined as having a wavelength in the range of about 700 nm-1400 nm. NIR fluorescent probes of the present invention are preferably those that absorb and emit NIR light in the range of about 700 to 900 nm, which is considered a biological “NIR window” as will be explained in more detail below.

Examples of suitable NIR fluorescent probes include dyes, e.g. cyanine dyes, such as indocyanine green (ICG), Cy5, Cy5.5, Cy5.18, Cy7 and Cy7.5; an IRDYE®, an ALEXA FLUOR® dye, a BODIPY® dye, an ANGIOSTAMP™ dye, a SENTIDYE™ dye, XENOLIGHT DIR™ fluorescent dye, VIVOTRACK™ NIR fluorescent imaging agent, KODAK X-SIGHT™ dyes and conjugates, DYLIGHT™ dyes. NIR quantum dots may also be utilized as probes (synthesis and functionalization of NIR quantum dots is described, for example, in Ma et al., 2010, Analyst, 135:1867-1877). Each possibility represents a separate embodiment of the invention.

A particular embodiment of a NIR fluorescent probe to be used with the nanoparticles of the present invention is indocyanine green (ICG).

The nanoparticles of the present invention may comprise up to about 10% (w/w) of the NIR fluorescent probe, for example up to about 5%, up to about 1%, up to about 0.5%, between about 0.005-5% (w/w) of the NIR fluorescent probe.

Labeling of particles with fluorescent probes is known in the art. Exemplary procedures are exemplified hereinbelow.

The nanoparticles of the present invention may comprise at least one magnetic probe detectable by magnetic resonance imaging (MRI), in addition to the NIR fluorescent probe.

In some embodiments, the magnetic probe is bound to the outer surface of the nanoparticles, either covalently or non-covalently. In other embodiments, the magnetic probe is contained embedded within the inner core of, or coated by, the nanoparticles.

Magnetic nanoparticles include particles that are permanently magnetic and those that are magnetizable upon exposure to an external magnetic field, but lose their magnetization when the field is removed. Materials that are magnetic or magnetizable upon exposure to a magnetic field that lose their magnetic properties when the field is removed are referred to as superparamagnetic material. Examples of suitable superparamagnetic materials include, but are not limited to, iron, mixed iron oxide (magnetite), or gamma ferric oxide (maghemite) as well as substituted magnetites that include additional elements such as zinc. Superparamagnetic particles may range in size from about 1 nm to about 20 nm, for example between about 1-10 nm, between about 5-20 nm.

Preparation of superparamagnetic particles, and also nanoparticles comprising such superparamagnetic particles can be performed by methods known in the art, for example, as described in De Cuyper et al., 1988, Eur Biophys J, 15:311-319. Additional methods are described, for example, in U.S. Pat. No. 7,175,912, U.S. Pat. No. 7,175,909 and US 20050271745. Exemplary procedures are provided hereinbelow.

The pharmaceutical compositions of the present invention are formulated for parenteral administration.

In some embodiments, the pharmaceutical composition is formulated for intravenous administration. In some embodiments, the pharmaceutical composition is formulated for intraarterial administration. In some embodiments, the pharmaceutical composition is formulated for trans-nasal administration. In some embodiments, the pharmaceutical composition is formulated for intrathecal administration. In some embodiments, the pharmaceutical composition is formulated for intra-orbital administration.

The pharmaceutical compositions provided by the present invention may be prepared by conventional techniques, e.g., as described in Remington: The Science and Practice of Phamiacy, 19^{th} Ed., 1995. The compositions can be prepared, e.g., by uniformly and intimately bringing the active ingredient, i.e., the particles of the invention as defined above, into association with a liquid carrier, a finely divided solid carrier, or both, and then, if necessary, shaping the product into the desired formulation. The compositions may be in liquid, solid or semisolid form and may further include pharmaceutically acceptable fillers, earners, diluents or adjuvants, and other inert ingredients and excipients.

As used herein, the term “pharmaceutically acceptable”, when referring to an ingredient within the pharmaceutical compositions of the present invention, such as a carrier, refers to a medium that does not interfere with the effectiveness of the activity of the main agent, and is not toxic to the host to which it is administered.

The pharmaceutical compositions of the invention may be, for example, in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

The concentration of the nanoparticles within the pharmaceutical composition of the present invention may range from about 0.01(w/w) to about 10% (w/w), for example in the range of about 0.05-5%, about 1 to 6% (w/w).

Methods and Uses

According to an aspect of the present invention, there is provided herein a method for detecting activated immune cells, such as activated phagocytic cells of the immune system, in the CNS of a subject. In some embodiments, a method for detecting areas of inflammation within the CNS of a subject is provided. As used herein the CNS includes the brain and spinal cord. Each possibility represents a separate embodiment of the invention.

As used herein, the term “detecting”, when referring to activated immune cells, refers to determining the presence or absence of the activated immune cells, and identifying the location of the activated immune cells, either qualitatively or quantitatively. The term may further refer to identifying signals from a probe and/or quantifying signals from a probe.

In some embodiments, the method comprises the steps of: (i) systemically administering to a subject via a parenteral route of administration a pharmaceutical composition of the present invention as described above, comprising nanoparticles labeled with a NIR fluorescent probe; (ii) irradiating at least a portion of the CNS of the subject with NIR radiation having a wavelength suitable for excitation of the NIR fluorescent probe; and detecting NIR fluorescence emission from the probe, wherein a locality of said fluorescence emission from the probe is indicative of a locality of activated immune cells, thereby detecting activated immune cells in the CNS of the subject.

In some embodiments, the method comprises detecting NIR fluorescence emission from a pre-administered NIR fluorescent probe. In some embodiments, the method comprises detecting the fluorescence of the pre-administered probe from a portion of the CNS of a subject following parenteral systemic administration of a pharmaceutical composition of the present invention comprising nanoparticles labeled with a NIR fluorescent probe, and irradiation of said portion of the CNS of a subject with NIR radiation that is suitable for excitation of the probe, wherein a locality of said fluorescence emission from the probe is indicative of a locality of activated immune cells, thereby detecting activated immune cells in the CNS of the subject.

In some embodiments, the nanoparticles are further labeled with a magnetic probe that is detectable by MRI. According to these embodiments, the method may further comprise a step of imaging using MRI. The locality of the signal collected from the magnetic probe is indicative of a locality of activated immune cells.

In some embodiments, there is provided herein a use of a pharmaceutical composition of the present invention, for the detection and imaging of immune cell activation in the CNS of a subject.

In some embodiments, there is provided herein a use of nanoparticles labeled with a NIR fluorescent probe as described above, for the manufacture of a pharmaceutical composition for detection and imaging of immune cells activation in the CNS of a subject.

The pharmaceutical composition containing the labeled nanoparticles is administered systemically via a parenteral route of administration. For example, the pharmaceutical composition may be administered by intravenous injection. As another example, the pharmaceutical composition may be administered by intranasal administration. As yet another example, the pharmaceutical composition may be administered by intraperitoneal administration.

The pharmaceutical composition may be administered several minutes up to several hours prior to the detection step. For example, it may be administered about 5-30 minutes prior to the detection step, about 5-20 minutes, about 10-20 minutes prior to the detection step. Alternatively, it may be administered 1-10 hours prior to the detection step, for example about 1-5 hours prior to the detection step.

Following administration of the labeled nanoparticles, NIR radiation is delivered to areas of the CNS to be examined, resulting in excitation of the NIR fluorescent probe and emission of fluorescent NIR radiation therefrome.

NIR radiation is typically defined as having a wavelength in the range of about 700 nm-1400 nm. For clinical applications, NIR light in the range of about 700 to 900 nm, is preferable, since within this range (sometimes referred to as the “NIR window”), absorption of most biomolecules (i.e., deoxyhemoglobin, oxyhemoglobin, water, and lipids) reaches local minima, scattering is relatively low, and tissue autofluorescence is relatively low. Thus, photon penetration into, and out of, tissue is relatively high (can penetrate depths such as several centimeters), and the use of an exogenous NIR fluorophore absorbing and emitting in this NIR window produces a high signal-to-background ratio (Gioux et al., 2010, Mol Imaging, 9(5):237-255; Miwa, 2010, The Open Surgical Oncology Journal, 2:26-28). The low energy of emission and excitation is biologically safe, compared, for example, with UV-excited compounds.

Thus, NIR fluorescent probes particularly suitable for use with the methods of the present invention are those characterized by excitation and emission light within the NIR window, for example excitation light in the range of about 700-850 nm, about 750-800 nm, and emission light in the range of about 750-850 nm, for example about 800-850 nm.

The wavelength of the NIR radiation that is applied by the method of the present invention is determined according to the selected NIR fluorescent probe. The wavelength is typically determined by the absorption maxima of the probe, which are known from scientific literature to a person of skill in the art. The emission maxima of a selected probe is also known from scientific literature to a person of skill in the art.

The emitted signals are captured by suitable equipment as will be further detailed below, and areas of activated immune cells, corresponding to areas of inflammation, are visualized. The target area to be scanned may include a portion of the CNS of the subject. For example, the target area may all of the brain or a specific area of the brain. In some embodiments, the scanned area is several centimeters wide, for example about 1-20 cm, 1-10 cm, 5-10 cm wide, but repeated screening of adjacent or non-adjacent areas can be performed. In some embodiments, continuous monitoring is performed over a period of time of at several minutes up to several hours.

Detection can be performed non-invasively, for example, by delivering IR radiation through the scalp and skull of the subject to at least one portion of the brain of the subject. Detection can also be performed intra-operatively. The detection device should be operated such that is captures the NIR radiation emitted from the probe, in the suitable wavelength, as known in the art.

In some embodiments, detecting comprises obtaining one or more images of the portion of the CNS irradiated by NIR where areas of NIR fluorescent emission are indicated. For example, a merged image of color and NIR images can be generated, showing the fluorescent areas marked within the colored image. In some embodiments, detecting comprises detecting using a microscope with appropriate filters.

Suitable devices for imaging according to embodiments of the present invention are commercially available, and include for example, surgical NIR fluorescent microscopes, e.g. Premium Surgical Microscope Leica M720 OHS, equipped with NIR-filters, e.g., Fluorescence module 820 nm/NIR Leica FL800. Another example of a commercially available surgical NIR fluorescent microscope is Zeiss OPMI Pentero (Carl Zeiss Surgical, GmbH, Germany), equipped, for example, with Zeiss INFRARED 800 module.

Additional examples of commercially available imaging systems that can be used with the methods of the present invention include the SPY™ imaging system (Novadaq Technologies Inc., Canada), HyperEye Medical System (Mizuho Medical Co. Ltd.), FLARE™ imaging system (the Beth Israel Deaconess Medical Center, USA), and Photodynamic Eye (PDE; Hamamatsu Photonics K.K., Japan).

The methodology of the present invention offers the capability of non-radioactive, simple imaging of regional and global immune cell function as a surrogate marker of CNS disease. The subject to be examined according to embodiments of the present invention may be a subject having, or suspected of having, a disease associated with CNS inflammation, namely, a disease affecting at least a portion of the CNS, which involves accumulation of activated phagocytic cells within the diseased CNS tissue. The subject is a mammal, typically a human.

For example, the subject may be an epilepsy patient. In some embodiments, the subject is an epilepsy patient that does not respond to medications (known as refractory epilepsy, or drug-resistant epilepsy). Epilepsy is a central nervous system disorder (neurological disorder) in which the nerve cell activity in the brain is disturbed, causing seizures, namely, episodes of disturbed brain activity during which the patient may experience abnormal behavior, symptoms and sensations, including loss of consciousness. Some forms of epilepsy are generalized, characterized by seizures that distort the electrical activity of the whole or a large portion of the brain. Other forms are partial (or focal), characterized by seizures that originate in a small, defined area of the brain (localized seizures may spread to larger portions of the brain following their generation). Treatment of epilepsy aims at reducing or eliminating the seizures. Treatment usually includes anti-epileptic drugs. Patients with refractory epilepsy are sometimes referred to surgery, to remove the part of the brain that triggers the seizures. Surgery is most often performed for refractory focal epilepsy, where the seizures originate in a small, well-defined area of the brain that does not interfere with vital functions like speech, language, motor function, vision or hearing. Before surgery, there is a need to locate the epileptic focus (the location of the epileptic abnormality) and to determine whether respective surgery will affect normal brain function. The evaluation typically includes neurological examination, routine electroencephalography (EEG), long-term video-EEG monitoring, neuropsychological evaluation, and neuroimaging such as MRI, single photon emission computed tomography (SPECT), positron emission tomography (PET), and sometimes functional MRI or magnetoencephalography (MEG) as supplementary tests. It would be highly advantageous to have means to image the area to be removed not only before the surgery, but also during the surgery.

Epilepsy has traditionally been considered mainly a neuronal disease. Only recently attention has been directed towards the role of the immune system in the pathophysiology of the disease (Vezzani el a., 2011, Nat Rev Neurol, 7:31-40; Zattoni et al., 2011, J Neurosci, 31:4037-4050). In particular, it is thought that areas of the brain that trigger the epileptic seizures are characterized by inflammation and accumulation of activated immune cells.

The method of the present invention proposes to use these activated immune cells as markers for the areas of inflammation in the brain of an epileptic subject. Following systemic parenteral administration of the composition comprising nanoparticles labeled with a NIR fluorescent probe, the particles undergo phagocytosis by immune cells found in the areas of inflammation. The immune cells may include peripheral phagocytes that infiltrated into the brain during the inflammatory process or resident active phagocytes, such as resident microglia. Irradiation of brain areas with NIR light suitable for excitation of the probe, and collection of fluorescence from the irradiated areas allow the identification of areas where activated phagocytic cells are found, and accordingly identification of possible epileptic foci. Advantageously, monitoring using the method of the present invention can be performed intra-operatively, for real-time inspection of an inflamed brain tissue in an epileptic subject, as the method can be practiced using equipment that is available in neurosurgery suites.

The subject to be examined by the methods of the present invention may be a subject having, or suspected of having, cerebral malaria.

Cerebral malaria is a neurological complication of infection with the malaria parasite (Plasmodium genus), involving brain inflammation. Clinical manifestations typically include fever, impaired consciousness, and in severe cases coma. Brain swelling, intracranial hypertension, retinal changes (hemorrhages, peripheral and macular whitening, vessel discoloration and or papilledema) and brainstem signs (abnormalities in posture, pupil size and reaction, ocular movements or abnormal respiratory patterns) are commonly observed. Early diagnosis of cerebral malaria may contribute to better treatment outcome. Detection of brain inflammation using the methods of the present invention may be useful in aiding the diagnosis of cerebral malaria.

Detection of CNS inflammation, such as brain inflammation, may also be useful as a complementary test for the diagnosis of other CNS disorders known to involve CNS inflammation, as well as for the evaluation of disease state. Sequential testing using the methods of the present invention may be used for monitoring the response of a subject to medical interventions.

The CNS disorders may include cysticercosis, an infection by the parasite Taenia solium, particularly neurocysticercosis, which is caused by cysts of the parasite in the brain. The CNS disorders may include lupus, where cerebritis commonly occurs. The CNS disorders may include multiple sclerosis, an inflammatory disease in which myelin sheaths around axons of the brain and spinal cord are damaged, leading to loss of myelin and scarring. The methods of the present invention may also be used for the assessment of brain inflammation in autoimmune encephalomyelitis, stroke, glioma, Alzheimer's disease and Parkinson's disease, for which brain inflammation is known to be involved. The methods of the present invention may also be employed for the assessment of brain inflammation following a traumatic brain injury.

In some embodiments, the disease is epilepsy. In some embodiments, the disease is epilepsy is cerebral malaria. In some embodiments, the disease is cysticercosis. In some embodiments, the disease is lupus. In some embodiments, the disease is multiple sclerosis.

In some embodiments, the disease is autoimmune encephalomyelitis. In some embodiments, the disease is stroke. In some embodiments, the disease is glioma. In some embodiments, the disease is Alzheimer's disease. In some embodiments, the disease is Parkinson's disease. In some embodiments, the disease is a traumatic brain injury. In some embodiments, the disease is autism. In some embodiments, the disease is schizophrenia.

The methods described herein may be combined with known methods for diagnosis and follow up of patients having CNS disorders.

The methods of the present invention may also find use in research applications, either in humans or animal models of particular diseases.

For example, the methods of the present invention may be utilized to study the nature of activated immune cells, the timing of cell activation with regard to disease process and BBB permeability to macromolecules, as well as the impact of various interventions of those processes, in various CNS disorders. The methods may also be utilized for investigating the role of phagocytic cells in neurophysiology and brain pathophysiology. The methods may also be applied for studying the cross-talk between neurons and immune cells in brain diseases, as well as in the healthy brain (e.g., during development and aging).

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Evaluation of ICG-Labeled NP Uptake by Murine Macrophages

Preparation of Fluorescence-Labeled Liposomes

Liposomes were prepared as follows: 260 mg of PHOSPHOLIPON® S75 and 65 mg of cholesterol were solubilized in 10 ml of a methanol:chlorophorm (1:1) mixture. Solvent was evaporated by means of rotary evaporator. The dry film was hydrated by 5 ml of phosphate buffer (pH 7, 5 mM), sucrose 9.3%. To obtain the final size, liposomes were extruded by 20 times passage through a 1 ml syringe extruder (Avanti) through membrane with pore size of 100 nm. Indocyanine green (ICG) (1 mM) was bound to the liposome nanoparticles (NP) by co-incubation for at least 1 h at 5° C. The ICG binding was based on electrostatic and hydrophobic interactions.

Preparation of PEGyleted Liposomes

PEGylated liposomes were prepared as described above, except that in addition to the phospholipids and cholesterol, 90 mg of DSPE-PEG-2000 was added to the solvent mixture of chlorophorm:methanol.

Results

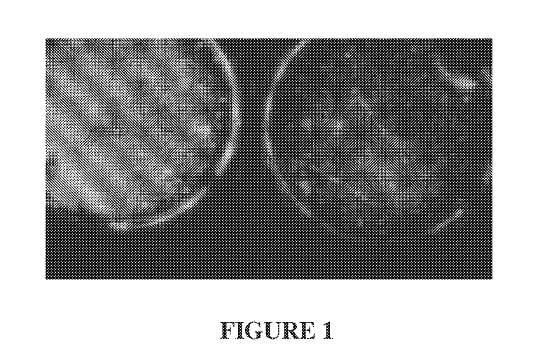

PEGylated or non-PEGylated NP labeled with ICG were prepared as described above. Macrophages of the RAW 264.7 cell line were incubated for 1 hr with NP and then washed. Petri dishes containing the macrophages were scanned by ODYSSEY® IR imaging system (LI-COR). FIG. 1 shows exemplary scans of Petri dishes containing macrophages incubated with non-PEGylated NP (left) or PEGylated NP (right). The uptake of non-PEGylated NP was 1.6 times greater compared to PEGylated NP.

### Example 2

### In Vivo Studies in Experimental Cerebral Malaria (ECM)

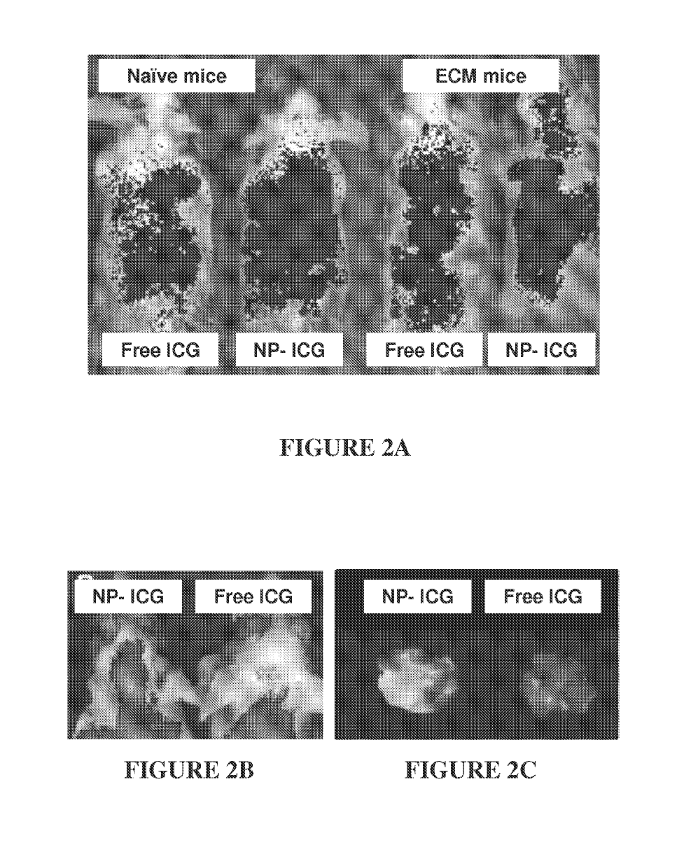

NP labeled with ICG (NP-ICG, non-PEGylated) prepared as described above or free ICG were injected into the tail vein of mice following infection with Plasmodium berghei ANKA or to naïve mice. Images of the mice were obtained 4 hours post injection. FIG. 2 shows exemplary images of in vivo (A, B) and ex-vivo (C) probe distribution into the CNS. As can be seen in the figure, in diseased mice, NP-ICG, but not free ICG, were preferentially uptaken into the brain of the mice. In naïve mice, no significant fluorescence was observed for NP-ICG or ICG in the brain of the mice.

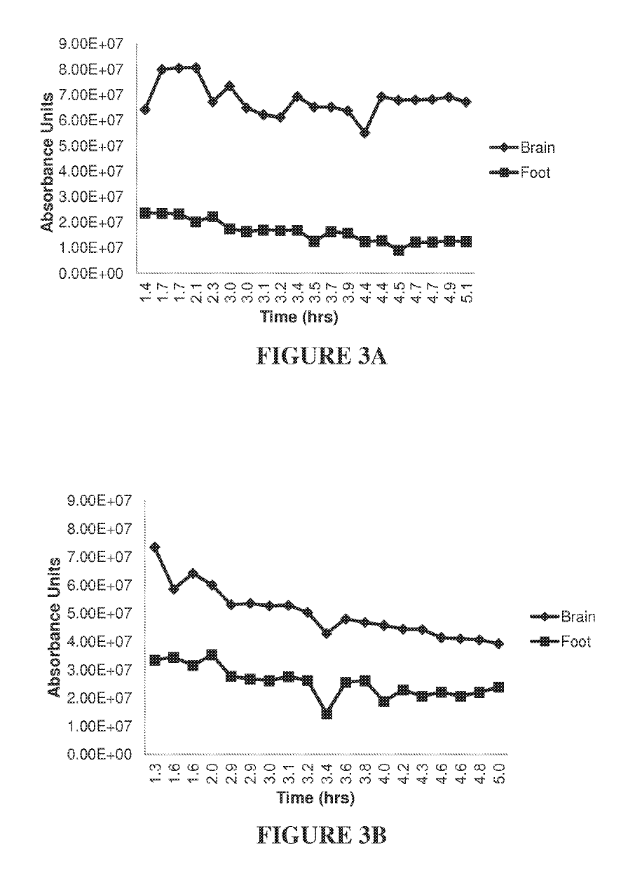

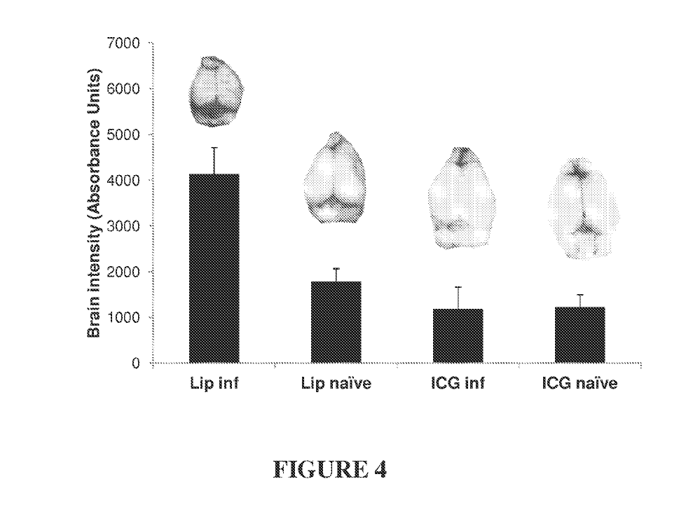

In an additional experiment, C57 black mice were infected with 80,000 parasites (P. berghei ANKA). Naïve mice and mice 6 days post infection were injected with NP-ICG. Mice were scanned by IVIS™ optical imager. The measurements were performed for 5 hours. For each mouse, the intensity of brain ICG emission was compared to that of the foot. FIG. 3 shows exemplary results of diseased (A) versus naïve (B) mice. Brain uptake of NP-ICG was 1.5 fold higher in diseased compared to naïve mice. In a further experiment, naïve mice and mice 6 days post infection with P. berghei ANKA were injected with NP-ICG or free ICG. Five hours post injection mice were sacrificed and brains were scanned by TYPHOON™ imager. The results are shown in FIG. 4. For NP-ICG, emission intensity of infected mice brains was 2.3 fold higher compared to normal mice (p<0.05). Free ICG was not statistically different for both groups.

### Example 3

### Characterization of Magneto-NP by Electron Microscopy

NP labeled with ICG and magnetite (magneto-NP) were prepared as described in De Cuyper et al., 1988, Eur Biophys J, 15:311-319 with several modifications: PHOSPHOLIPON® 50 was solubilized in a methanol:chloroform mixture 1:1. The solvents were evaporated and the resultant lipid film was hydrated to form multilamellar NP. The final size of the NP was controlled by using an extruder with submicron pore size. For magnetite preparation, ferrous chloride (FeCl₂) and ferric chloride (FeCl₃) salts were precipitated with excess of ammonia, and the precipitate was then washed with diluted ammonia solution. The precipitate was heated to 90° C. for 4 min, meanwhile lauric acid was added and finally diluted by water (pH 9). The binding of the organic nanoparticles and the magnetic ones is based on electrostatic interactions. The binding was performed by incubating the NP with the magnetic particles in dialysis tubes for 48 h against buffer solution. Unbound NP was magnetically separated. ICG was bound to the NP by co-incubation for at least 1 h at 5° C. The ICG binding is based on electrostatic and hydrophobic interactions. The unbound ICG was separated by ultrafiltration as described in Portnoy et al., 2011, Nanomedicine (Lond), 7:480-488. For future clinical use, NP can be sterilized by filtration.

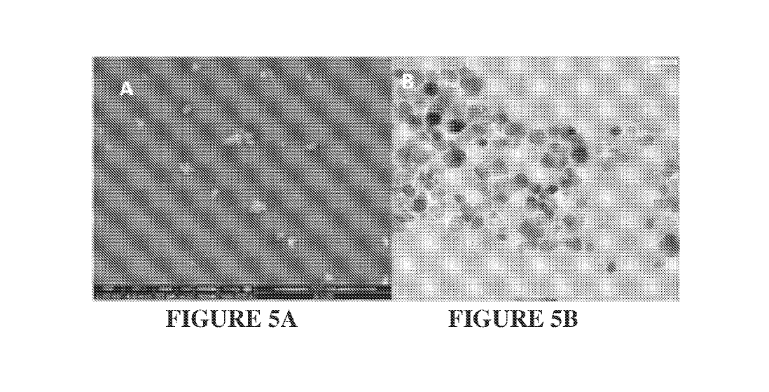

Magneto-NP were characterized by high resolution scanning electron microscopy (HR-SEM) and transmission electron microscopy (TEM). The resulting images of the NP (70-80 nm) are shown in FIGS. 5A and 5B, respectively. FIG. 5A shows magnetite (5-7 nm) as small aggregates (white particles) accumulated in an organic matter which surrounds the aggregates (dark spots). In FIG. 5B, only magnetite can be seen.

### Example 4

### In Vivo Studies in Epilepsy Rat Model Using PLA-Magnetite-BODIPY® Nanoparticles

Preparation of polylactic acid (PLA)-magnetite-BODIPY® nanoparticles Magnetite was obtained from ferric and ferrous chloride by alkaline precipitation as described in MacDonald et al., 2010, Nanomedicine, 5:65-76. Precipitated magnetite was magnetically separated, washed twice with degassed DI water, re-suspended in 2 mL of ethanol, and coated with 200 mg oleic acid with heating under argon to 90° C. in a water bath for 10 min. Excess oleic acid was phase-separated by drop-wise addition of 4 mL of water and the lipid-coated magnetite was washed twice with ethanol to remove the excess of the oleic acid. The lipophilic magnetite was dispersed in 6 mL of chloroform, forming stable magnetic fluids further used for nanoparticle preparation, where the lipophilic magnetite is loaded within a (poly)lactic acid (PLA) matrix.

Fluorescently labeled PLA were obtained as follows: carboxyl end groups of PLA were coupled with amine-containing BODIPY® using carbodiimide chemistry in organic medium. The carboxyl group activation step was carried out in methylene chloride using NHS/DIC at a 1:1 molar ratio to obtain succinimidyl ester of PLA (PLA-Su). The molar ratio of NHS/DIC to PLA was kept at 300. The activated PLA-Su was precipitated three times from methylene chloride into cold methanol. In the next step, the PLA-Su was coupled with BODIPY® under argon atmosphere for 24 h at basic conditions in methylene chloride supplemented with triethylamine. The excess of base was neutralized with acetic anhydride and the fluorescently labeled PLA was precipitated three times from methylene chloride into cold methanol.

Fluorescent PLA-based magnetic particles were formulated by dissolution of 180 mg of non-labeled PLA and 20 mg of fluorescently labeled (BODIPY®) PLA in 6 mL of magnetic fluid to form an organic phase. organic phase was emulsified in 15 mL of pre-chilled 1.5% (w/v) polyvinyl alcohol (PVA) by sonication, and the organic solvents were removed by evaporation under reduced pressure at 30° C. The particles obtained were passed through a 1.0 μm glass fiber and lyophilized with 10% (w/v) trehalose as a cryoprotectant. Lyophilized particles were kept at +4° C. in 100 μL aliquots and re-suspended in deionized water before use (see MacDonald et al., 2012, Pharm Res., 29(5):1270-81).

Results

Magnetite nanoparticles coated by polylactic acid conjugated to BODIPY® 660 were injected to tail vein of epileptic Wistar rats (2 months post initiation of epilepsy). The rats were sacrificed 4 and 24 hours post injection.

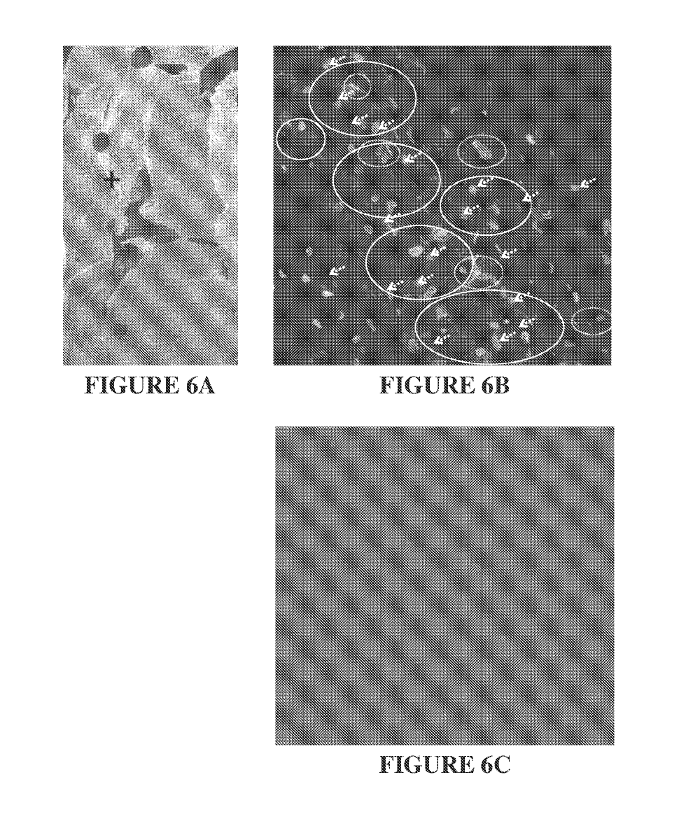

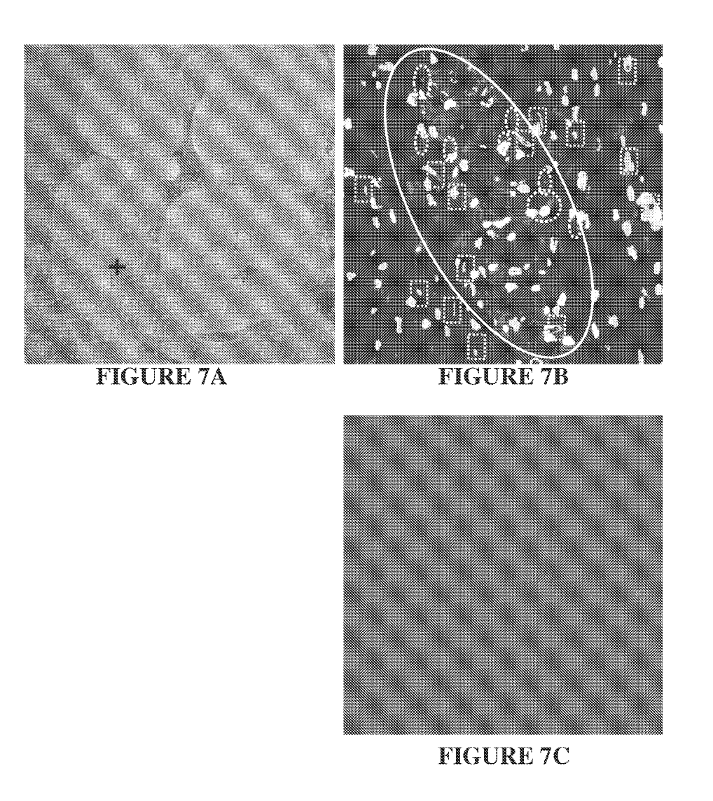

FIG. 6 shows confocal microscope images of an exemplary epileptic rat brain slice focused on epileptogenic brain region—hippocampus. The slice was stained for micro glia/macrophages (IBA1 or OX-42 red stain), astrocyte stain (GFP green stain) and DAPI cyan stain. FIG. 6A shows the stained brain slice with a “+” sign indicating the brain region which is illustrated in FIGS. 6B-C. FIG. 6B shows microglia/macrophages, astrocytes, DAPI and nanoparticles. Nanoparticles only are shown in FIG. 6C. The main points of nanoparticle localization (originally blue color) are indicated by dashed arrows. Main areas of microglia/macrophages (originally red staining) are indicated by dashed circles. Solid-line circles indicate the main areas of astrocytes (originally green staining). The nanoparticles mainly co-localized with microglia/macrophage stain and less with astrocytes, thus supporting specific uptake by myeloid immune cells. The specific uptake was further supported by co-stain of the microglia/macrophage marker with an endothelial cells marker (RECA-1green), showing co-localization of the particles mainly with microglia/macrophages and less with endothelial cells (FIG. 7A, stained brain slice with a “+” sign indicating the brain region which is illustrated in the next two figures; FIG. 7B, microglia/macrophages, endothelial cells, DAPI and nanoparticles. The main area of microglia/macrophages (originally red staining) is indicated by a solid-line circle, main points of nanoparticle localization (originally blue color) are indicated by dashed circles, and main points of endothelial cells (originally green staining) are indicated by dashed squares; FIG. 7C, nanoparticles only).

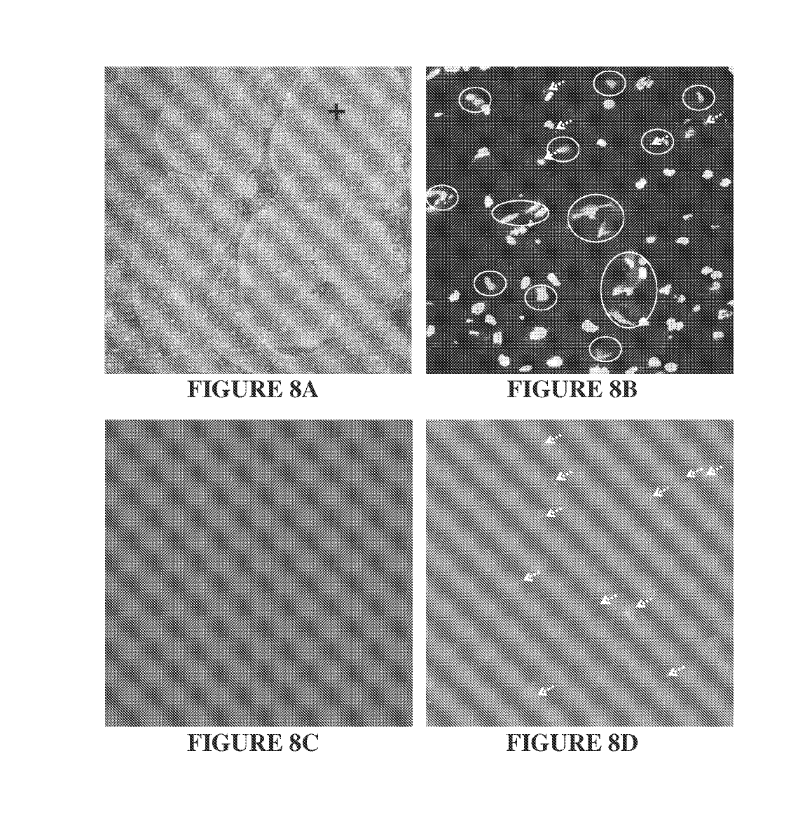

Staining of the thalamus, chosen as a reference region close to the hippocampus, showed minimal staining and co-localization of microglia/macrophages with nanoparticles (FIG. 8A, stained brain slice with a “+” sign indicating the brain region which is illustrated in the next three figures; FIG. 8B, microglia/macrophages, endothelial cells, DAPI and nanoparticles, main areas of endothelial cells (originally green staining) are marked by circles, main points of nanoparticles (originally blue color) are indicated by dashed arrows; FIG. 8C, nanoparticles only; FIG. 8D, nanoparticles and microglia/macrophages, main points of nanoparticles (originally blue color) are indicated by dashed arrows).

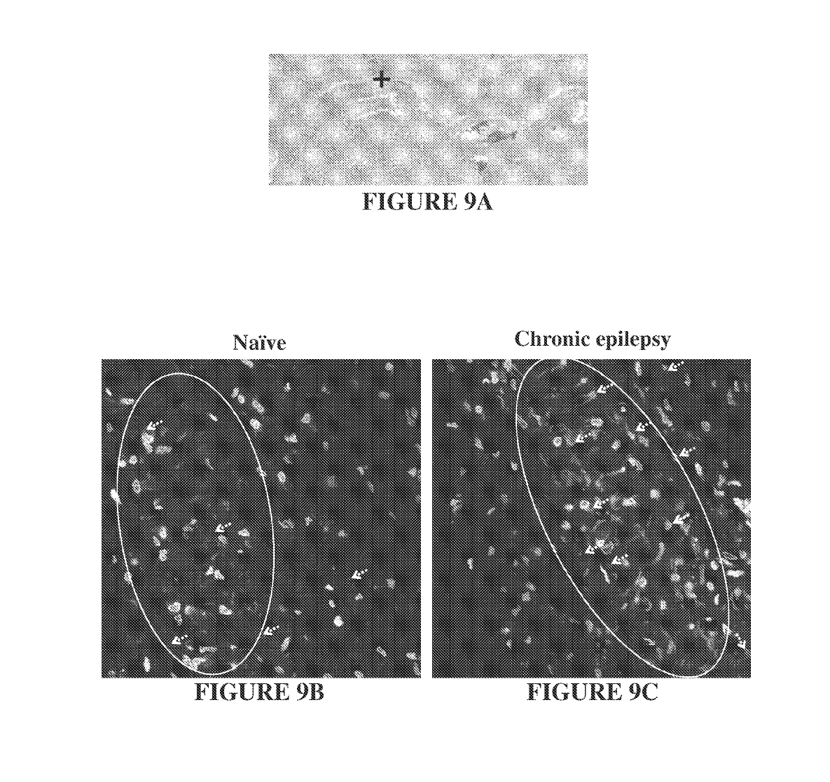

The brain slices of epileptic rats sacrificed 4 h post injection of nanoparticles were compared to brain slices of naïve rats 4 h post injection of nanoparticles. Slices were stained for microglia/macrophages and DAPI. FIG. 9 shows an exemplary comparative staining. As can be seen in the figure, fewer particles were observed in the hippocampus of a naïve rat compared to the hippocampus of an epileptic rat (FIG. 9A, stained brain slice with a “+” sign indicating the brain region which is illustrated in the next two figures; FIG. 9B, naïve rat, FIG. 9C, epileptic rat. Main areas of microglia/macrophages (originally red staining) are indicated by circles, main points of nanoparticles (originally blue color) are indicated by dashed arrows).

### Example 5

### Uptake of Neutral Versus Negatively Charged PLA-Based Particles by Murine Macrophages

Neutral PLA-based nanoparticles were formulated using emulsification-evaporation method with incorporation of oleic acid coated magnetite crystals within the polymer core as described in MacDonald et al., 2010 noted above, Macdonald et al., 2012 noted above and Johnson et al., 2010, Current drug delivery, 7:263-273, using 1.5% (w/v) poly(vinyl alcohol) (PVA) as a stabilizer agent during the emulsification step. The mean hydrodynamic diameter of these particles was around 280 nm with polydispersity index of 0.161. The surface charge (zeta potential) of these particles was in the range of −6-9 mV (which is considered neutral).

Negatively charged nanoparticles based on a surface functionalized polymer bearing negative charge by carboxylic groups were prepared by the same method utilizing 1.25% (w/v) PVA and 0.25% (w/v) of the polymer. The negatively charged particles had a mean hydrodynamic diameter of 332 nm with polydispersity index of 0.189. The zeta potential of these particles was −28-32 mV.

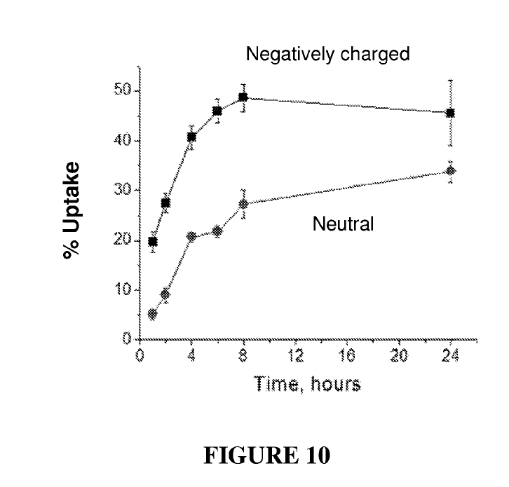

Both neutral and negatively charged particles contained 50% (w/w) magnetite and a fluorescent label (BODIPY®) covalently linked to PLA as described above. Nanoparticle uptake was studied on adhered murine macrophage cell line (RAW 264.7). The results have shown that the phagocytic cells internalized negatively charged nanoparticles about 2.5-fold more efficiently comparing to the neutral nanoparticles (45% vs. 20% uptake at 6 hours respectively, FIG. 10).

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A method for detecting activated phagocytic cells in the central nervous system (CNS) of a subject, the method comprising the steps of:
(i) parenterally administering to a subject a pharmaceutical composition comprising nanoparticles configured for enhanced phagocytosis by phagocytic cells, the nanoparticles characterized by at least one structural or physicochemical feature that enhances their uptake by phagocytic cells compared to equivalent nanoparticles without the at least one structural or physicochemical feature, the nanoparticles further comprising a near-infrared (NIR) fluorescent probe;
(ii) irradiating at least a portion of the CNS of the subject with NIR radiation having a wavelength that is absorbed by the NIR fluorescent probe; and
(iii) detecting NIR fluorescence emission from the probe, wherein a locality of said fluorescence emission from the probe is indicative of a locality of activated phagocytic cells, thereby detecting activated phagocytic cells in the CNS of the subject.

2. The method of claim 1, wherein the size of the nanoparticles is in the range of about 80 nm-20 microns.

3. The method of claim 2, wherein the size of the nanoparticles is in the range of about 80 nm-1000 nm.

4. The method of claim 1, wherein the nanoparticles are charged nanoparticles.

5. The method of claim 1, wherein the nanoparticles comprise at least one targeting moiety bound to an outer surface of the nanoparticles, the moiety targeting the nanoparticles to phagocytic cells.

6. The method of claim 1, wherein the at least one structural or physicochemical feature of the nanoparticles is least one of: size in the range of about 80 nm-20 microns, negative or positive charge, and a surface-bound targeting moiety that targets the nanoparticles to phagocytic cells.

7. The method of claim 1, wherein the NIR fluorescent probe is a fluorescent dye or NIR quantum dots.

8. The method of claim 1, wherein the NIR fluorescent probe is bound to an outer surface of the nanoparticles.

9. The method of claim 1, wherein the NIR fluorescent probe is embedded within the nanoparticles.

10. The method of claim 1, wherein the nanoparticles further comprise at least one magnetic probe detectable by magnetic resonance imaging (MRI).

11. The method of claim 1, wherein the nanoparticles are capable of penetrating the blood-brain-barrier.

12. The method of claim 1, wherein the nanoparticles are selected from the group consisting of liposome nanoparticles, polymeric nanoparticles and solid lipid nanoparticles.

13. The method of claim 1, wherein the step of detecting comprises obtaining one or more images of the portion of the CNS irradiated by NIR where areas of NIR fluorescent emission are indicated.

14. The method of claim 1, wherein the step of detecting NIR fluorescence emission from the probe comprises using a microscope with appropriate filters.

15. The method of claim 1, wherein the NIR radiation has a wavelength in the range of about 700-850 nm.

16. The method of claim 1, wherein the subject is having, or suspected of having, a disease associated with CNS inflammation.

17. The method of claim 16, wherein the disease associated with CNS inflammation is selected from the group consisting of epilepsy, cerebral malaria, cysticercosis, lupus, multiple sclerosis, autoimmune encephalomyelitis, stroke, glioma, Alzheimer's disease, Parkinson's disease, traumatic brain injury, autism, and schizophrenia.

18. The method of claim 1, wherein the method is used to detect phagocytic cells in an area of inflammation in the brain of the subject.

19. The method of claim 1, wherein the step of administering the pharmaceutical composition further comprises administration via a route of administration selected from the group consisting of intravenous, intraarterial, trans-nasal, intrathecal, and intra-orbital administration.

20. The method of claim 1, wherein the step of detecting NIR fluorescence emission from the probe is performed several minutes up to several hours following the step of administering of the pharmaceutical composition.
